(19) United States
(12) Patent Application Publication
(10) Pub. No.: US 2009/0145754 A1
(43) Pub. Date: Jun. 11, 2009

(54) BIOCHEMICAL TEST SYSTEM, MEASUREMENT DEVICE, BIOCHEMICAL TEST STRIP AND METHOD OF MAKING THE SAME

(75) Inventors: Mon Wen Yang, Hsnchu City (TW); Ching-Yuan Chu, Hsinchu City (TW); Yueh-Hui Lin, Hsinchu City (TW); Ming-Chang Hsu, Hsinchu City (TW); Jui-Ping Wang, Hsinchu City (TW); Thomas Y.S. Shen, Hsinchu City (TW)

Correspondence Address: SNELL & WILMER L.L.P. (Main), 400 EAST VAN BUREN, ONE ARIZONA CENTER, PHOENIX, AZ, 85004-2202, US

(73) Assignee: APEX BIOTECHNOLOGY CORP., Hsinchu City (TW)

(21) Appl. No.: 12/038,932

(22) Filed: Feb. 28, 2008

(30) Foreign Application Priority Data
Dec. 7, 2007 (TW) 96146711

(51) Int. Cl. G01N 33/50

(52) U.S. Cl. 204/403.02; 427/356

(57) ABSTRACT

A biochemical test system, a measurement device, a biochemical test strip and a method of making the same are provided. The biochemical test system includes a biochemical test strip and a measurement device. The biochemical test strip includes an insulating substrate, an electrode system disposed on the insulating substrate, and a pattern code disposed on one side of the insulating substrate. The pattern code includes N components and at least one of the N components penetrates the insulating substrate. The measurement device includes a microprocessor and a connector. The connector is coupled to the pattern code and the electrode system for receiving signals corresponding to the pattern code. The microprocessor is coupled to the connector for receiving signals from the connector.

### CROSS REFERENCE TO RELATED APPLICATIONS

This application claims the right of priority based on Taiwan Patent Application No. 096146711 entitled “BIOCHEMICAL TEST SYSTEM, MEASUREMENT DEVICE, BIOCHEMICAL TEST STRIP AND METHOD OF MAKING THE SAME,” filed on Dec. 7, 2007, which is incorporated herein by reference and assigned to the assignee herein.

### FIELD OF INVENTION

The present invention relates to a biochemical test system, a measurement device, a biochemical test strip, and a method of making the same, and more particularly, to a biochemical test system, a measurement device, a biochemical test strip, and a method of making the same, with an auto correction to dismiss the need for a code card.

### BACKGROUND OF THE INVENTION

With the advance of the medical science and the rising concept from the modem people about health care, the Point-of-Care (POCT) has been widely available to the market. Such kinds of self-testing products, such as blood glucose monitor, electrical ear thermometer, and electrical sphygmomanometer, tend to be fast, cheap, and small and generally do not require professional help for the operation. In such field, the use of the biochemical test strip is a well-versed skill, especially for the popular application of monitoring blood glucose.

In the conventional biochemical test system, every batch of biochemical test strips has been defined a unique parameter during the production process. Therefore, before using a batch of biochemical test strip for a test on a measurement device, a code card is needed to calibrate the measurement device, as disclosed in U.S. Pat. No. 5,582,697 and PCT Publication No. WO00/33072. However, to manufacture the code card will increase the production cost and the labor associated with using the system; also correction error and data measurement error occur frequently because users forget to insert the code card, or use a wrong code card, or the code card is lost.

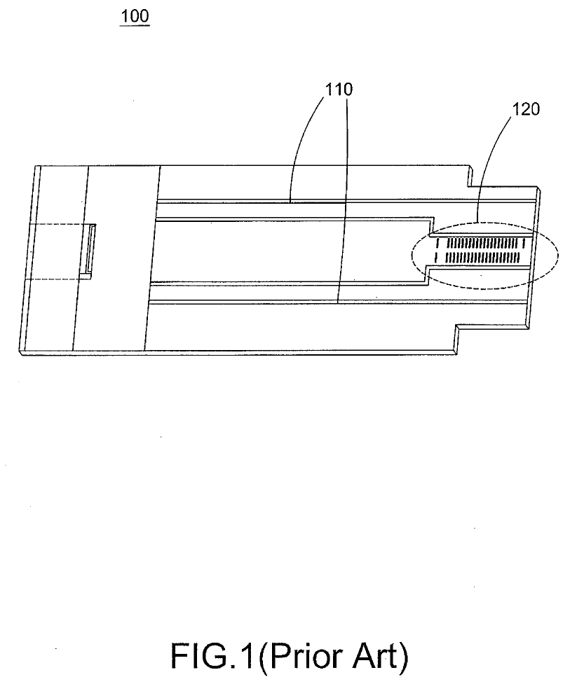

To solve the inconvenience with using the code card, U.S. Pat. No. 6,814,844 disclosed an identification method by using bar codes. FIG. 1 indicates a conventional test strip 100 comprising a conductive electrode set 110 with a plurality of electrodes insulated from each other, and a bar code 120 disposed between the conductive electrode set 110. The bar code 120 is a bar code pattern formed on the substrate by laser ablation; specifically, the bar code pattern is formed by using a high-energy pulsed laser to bombard the surface of a gold target material coated on the substrate, so that a portion of the gold target material is removed, and the desired bar code pattern is formed. However, as disclosed in U.S. Pat. No. 6,814,844, the identification methods for bar code 120 are optical measurement systems, using CCD or LED for detection, for example. Moreover, the reproduction and the accuracy highly depend on the surface condition of the target material, therefore there is not only a limitation to the fabrication, but also an increase in the production cost.

Accordingly, it is advantageous to have a biochemical test system capable of avoiding the code card correction and keeping the production yield and the test accuracy.

### SUMMARY OF THE INVENTION

In view of the problems existing in the prior art, the present invention provides an auto-correction biochemical test system capable of eliminating the use of a discrete code card, and reducing the production failure rate.

According to an aspect of the present invention, a biochemical test system including a biochemical test strip and a measurement device is provided. The biochemical test strip includes an insulating substrate, an electrode system disposed on the insulating substrate, and a pattern code disposed on one side of the insulating substrate. The pattern code includes N components, and at least one of the N components penetrates the insulating substrate. It should be noted that the term “N” in this specification is a positive integer. The measurement device includes a microprocessor and a connector, wherein the connector is coupled to the pattern code and the electrode system for receiving a signal corresponding to the pattern code, and the microprocessor is coupled to the connector for receiving the signal from the connector.

According to another aspect of the present invention, a measurement device is provided. The measurement device is used with a biochemical test strip, wherein the biochemical test strip includes an insulating substrate, an electrode system disposed on the insulating substrate, and a pattern code disposed on one side of the insulating substrate. The pattern code includes N components, and at least one of the N components penetrates the insulating substrate. The measurement device includes a connector electrically coupled to the pattern code for receiving a signal corresponding to the component of the pattern code penetrating the insulating substrate, and the measurement device includes a microprocessor coupled to the connector for receiving the signal from the connector.

According to another aspect of the present invention, a biochemical test strip including an insulating substrate, an electrode system disposed on the insulating substrate and a pattern code disposed on one side of the insulating substrate is provided. The pattern code includes N components and at least one of the N components penetrates the insulating substrate. A plurality of different identification codes can be composed by respectively controlling each of N components to penetrate the substrate or not.

According to another aspect of the present invention, a method for producing a biochemical test strip is provided. The method includes the following steps: (a) providing an insulating substrate; (b) forming a conductive layer on the insulating substrate; (c) providing an insulating layer on the conductive layer, wherein the insulating layer exposes a part of the conductive layer to define a reaction area with an opening; (d) providing a cover on the insulating layer, wherein the cover at least covers the reaction area; (e) performing a cutting or a punching process to produce a plurality of biochemical test strips each respectively having a predefined pattern code, wherein each of the predefined pattern code has a component penetrating the insulating substrate.

The other aspects of the present invention, part of them will be described in the following description, part of them will be apparent from description, or can be known from the execution of the present invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE PICTURES

The foregoing aspects and many of the attendant advantages of this invention will become more readily appreciated as the same becomes better understood by reference to the following detailed description, when taken in conjunction with the accompanying pictures, wherein:

FIG. 1 illustrates a conventional biochemical test strip;

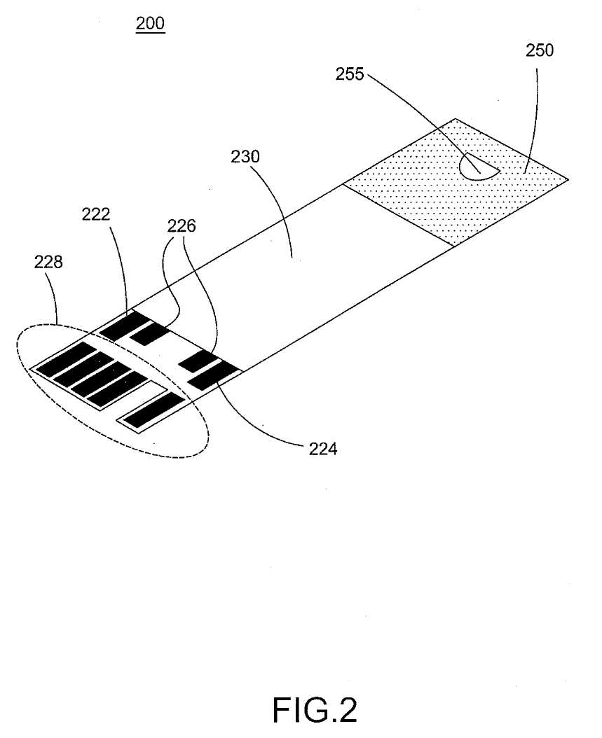

FIG. 2 illustrates a biochemical test strip according to an embodiment of the present invention;

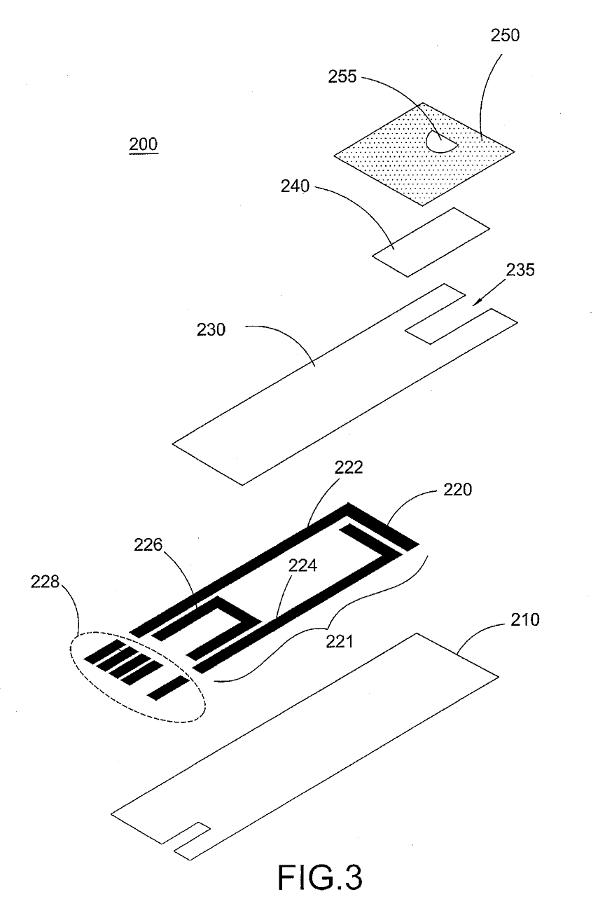

FIG. 3 illustrates an explosive view of the biochemical test strip shown in FIG. 2;

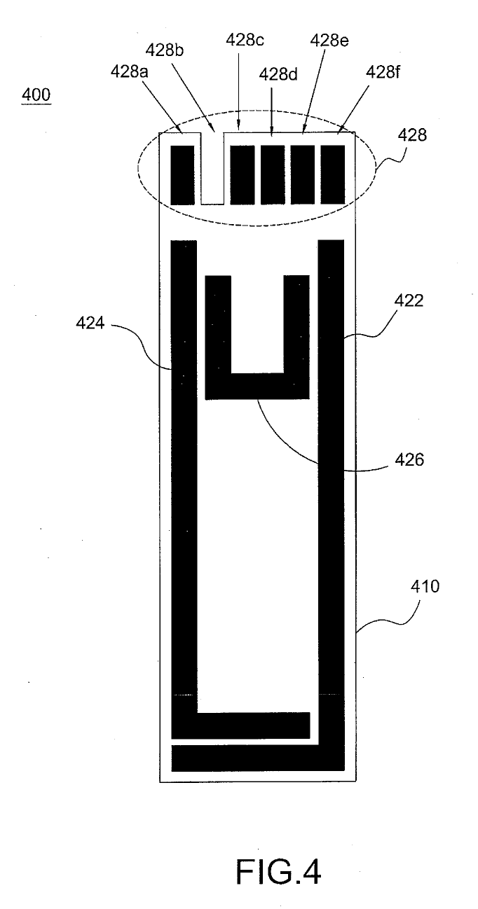

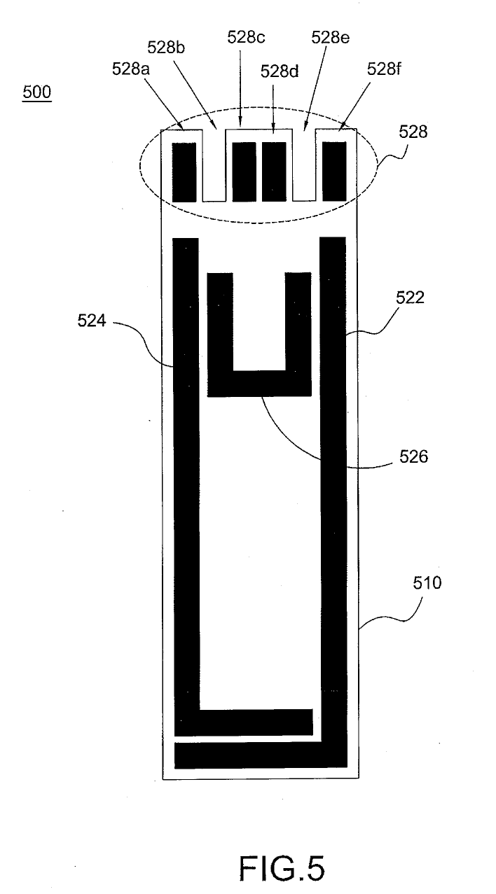

FIGS. 4 and 5 are the biochemical test strips according to different embodiments of the present invention;

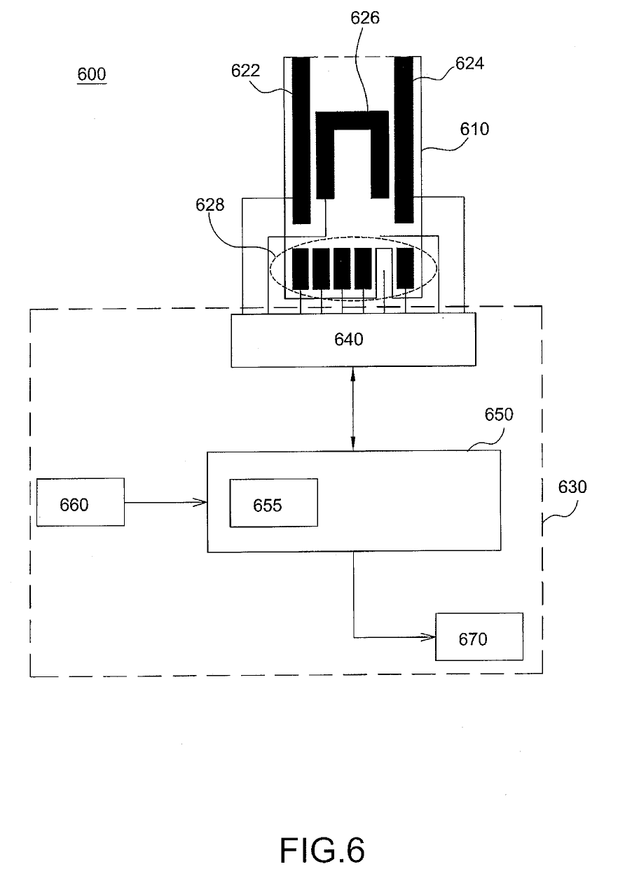

FIG. 6 is a block diagram of a biochemical test system according to an embodiment of the present invention; and

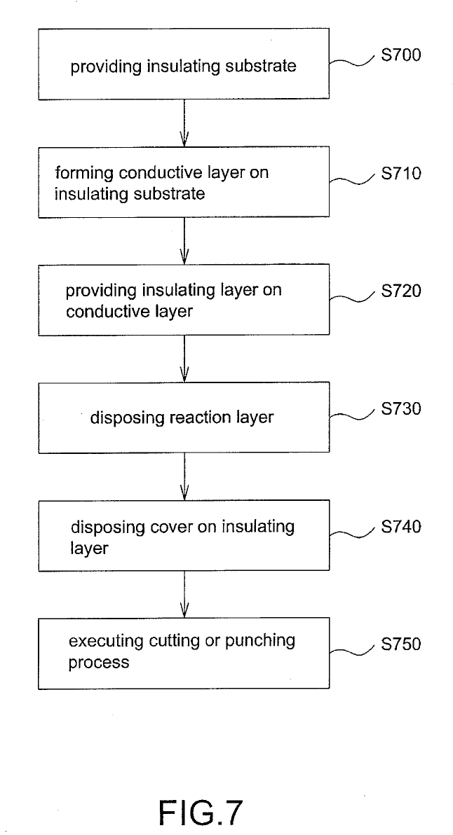

FIG. 7 is a flow chart of producing a biochemical test strip according to an embodiment of the present invention.

### DETAILED DESCRIPTION OF THE INVENTION

The present invention discloses a biochemical test system, a measurement device, a biochemical test strip, and a method of making the same, which eliminate the need of a discrete code card, provide easy operation, prevent the users from forgetting to insert the code card or use a wrong code card, and reduce the possibility of errors during the production process. The present invention will be described more fully hereinafter with reference to the FIGS. 2-7. However, the devices, elements, and methods in the following description are configured to illustrate the present invention, and should not be construed in a limiting sense.

FIG. 2 illustrates a biochemical test strip 200 according to an embodiment of the present invention, and FIG. 3 illustrates an explosive view of the biochemical test strip 200 shown in FIG. 2. The biochemical test strip 200 of the present invention includes an insulating substrate 210, a conductive layer 220, an insulating layer 230, and a cover 250. The conductive layer 220 includes an electrode system 221 and a pattern code 228, wherein the pattern code 228 includes a part penetrating the insulating substrate 210. It should be understood that the pattern code 228 includes conductive material. In an embodiment, the electrode system 221 includes a working electrode 222, a reference electrode 224, and a sense electrode 226, insulated from one another.

The insulating substrate 210 is electrically insulating, and its material can include but not limit to: polyvinylchloride (PVC), glass fiber (FR-4), polyester, bakelite, polyethylene terephthalate (PET), Polycarbonate (PC), polypropylene (PP), polyethylene (PE), polystyrene (PS), or ceramic material.

The conductive layer 220 can be any known conductive material such as carbon paste, gold-silver paste, copper paste, carbon/silver paste, or other similar material and the combination thereof. In an embodiment, the conductive layer 220 includes a conductive silver paste layer and a conductive carbon paste layer disposed on the conductive silver paste layer. In this embodiment, the sense electrode 226 is disposed between the working electrode 222 and the reference electrode 224 and configured to detect an electrical connection between the biochemical test strip 200 and a measurement device (as 630 shown in FIG. 6). When the biochemical test strip 200 is inserted into the measurement device, a loop is formed between the sense electrode 226 and the measurement device to activate the measurement device. In fact, it's sufficient as each electrode in a reaction area follows the arrangement order as mentioned above, and each electrode is insulated from one another. The present invention is not limited to the arrangement method for the working electrode 222, the reference electrode 224 and the sense electrode 226 illustrated in the embodiment, or the number of electrodes used. Additional electrodes can be added according to different application need.

The insulating layer 230 is disposed on the conductive layer 220, and includes an indentation 235 to expose a part of the conductive layer 220. It's sufficient for the indentation 235 to expose part of the working electrode 222 and part of the reference electrode 224. The present invention is not limited to the shape of the indentation 235. Besides, the insulating layer 230 also exposes another part (not shown) of the conductive layer 220 so that the conductive layer 220 can electrically connect to the measurement device (as 630 shown in FIG. 6). The material of the insulating layer 230 can include but is not limited to: PVC insulating tape, PET insulating tape, thermal drying insulating paint or ultraviolet drying insulating paint.

The cover 250 is disposed on the insulating layer 230, covering the indentation 235. The indentation 235 forms a sample space (i.e. reaction area) with capillary attraction between the insulating substrate 210 and the cover 250. When the area of the sample space is fixed, its volume depends on the thickness of the insulating layer 230. Generally, the thickness of the insulating layer 230 is between 0.005 and 0.3 millimeter, but not limited thereto. Furthermore, an insulating layer 230 with a precut indentation 235 can be disposed on the insulating substrate 210 and the conductive layer 220. Alternatively, the insulating layer 230 can be formed directly on part of the insulating substrate 210 and the conductive layer 220 by a printing method, which is defined with the indentation 235 and exposes the contact area to be coupled with the measurement device.

The biochemical test strip 200 of the present invention further includes a reaction layer 240 with the ability to identify specified organism material or signal. The material of the reaction layer 240 can be varied with sample types, such as an oxidoreductase for reacting with the sample. Generally, the reaction layer 240 should at least cover part of the working electrode 222.

The cover 250 of the present invention can be transparent or translucent material, so that the users may check whether the sample has been disposed on the reaction area to avoid a false result. The lower surface of the cover 250 close to the reaction area can be coated with a hydrophile material to enhance the capillary action on the inner surface of the reaction area. In this way the sample can be conducted to the reaction area more quickly and efficiently. The cover 250 further includes a vent 255 corresponding to the reaction area for expelling the air inside the reaction area to enhance the capillary action. Generally, the vent 255 is near the end side of the reaction area. The present invention is not limited to the shape of the vent 255. For example, the vent 255 can be a circle, an ellipse, a rectangle, and a rhombus etc.

FIGS. 4 and 5 are biochemical test strips 400 and 500 according to different embodiments of the present invention. The biochemical test strip 400 includes an insulating substrate 410, a working electrode 422, a reference electrode 424, a sense electrode 426, and a pattern code 428, insulated from one another. The pattern code 428 includes six components: 428a, 428b, 428c, 428d, 428e, and 428f. In this embodiment, the component 428b is a groove penetrating the insulating substrate 410. Referring to FIG. 5, the biochemical test strip 500 includes an insulating substrate 510, a working electrode 522, a reference electrode 524, a sense electrode 526, and a pattern code 528, insulated from one another. The pattern code 528 includes six components: 528a, 528b, 528c, 528d, 528e, and 528f. In the embodiment of FIG. 5, the components 528b and 528e are grooves penetrating the insulating substrate 510. When the biochemical test strip 400 or 500 is inserted into the measurement device, the grooves (such as components 428b, 528b, or 528e) of the pattern codes can't form an electrical connection with the measurement device, therefore the biochemical test strip 400 or 500 can be identified by the measurement device.

Although FIGS. 4 and 5 indicate a pattern code with six components, the present invention is not limited to the number of the components. It should be understood that the number and the location of the grooves can be defined by the designer according to practical applications to compose a plurality of different identification codes. For example, 2^{N}-1 identification codes can be composed for a pattern code with N components. In another embodiment, the components 428b, 528b, or 528e can be holes penetrating the insulating substrate rather than the grooves. The present invention is not limited to the shape and size of the groove or the hole.

FIG. 6 is a block diagram of a biochemical test system 600 according to an embodiment of the present invention, including a biochemical test strip 610 and a measurement device 630. The biochemical test strip 610 includes a working electrode 622, a reference electrode 624, a sense electrode 626, and a pattern code 628. The pattern code 628 includes at least one groove, as mentioned above. The measurement device 630 includes a connector 640 and a microprocessor 650 coupled to the connector 640. A digital data 655, for example, testing parameters, detection modes or other information, are built in the microprocessor 650. The working electrode 622, the reference electrode 624, the sense electrode 626, and the pattern code 628 electrically connect to the measurement 630 through the connector 640.

When the biochemical test strip 610 is connected to the connector 640, a loop is formed between the sense electrode 626 and the connector 640 to initiate the microprocessor 650 of the measurement device 630. Furthermore, since the groove part of the pattern code 628 can't form an electrical connection with the connector 640, an open-circuit signal corresponding to the groove part can be identified by the microprocessor 650. The microprocessor 650 will process the identification according to the signal and choose testing parameters or a test mode from the digital data 655 corresponding to the signal for execution. The measurement device 630 further includes a monitor 670 to display each measurement result, and a power source 660 to provide necessary power. In another embodiment, the monitor 670 and the power source 660 can be external devices, not included in the measurement device 630.

FIG. 7 is a flow chart for manufacturing a biochemical test strip according to an embodiment of the present invention. First, in step S700, an insulating substrate is provided. Then, in step S710, a conductive layer is formed on the insulating substrate by coating the substrate with a conductive material. The conductive layer includes a plurality of electrodes insulated from one another and an undefined pattern code. The undefined pattern code indicates the pattern without groove or hole component, i.e. the pattern without the component 428b shown in FIG. 4 or the components 528b and 528e shown in FIG. 5. For example, in one embodiment the undefined pattern code can include components without groove or hole, such as the components 428a, 428c, 428d, 428e, and 428f shown in FIG. 4, or the components 528a, 528c, 528d, and 528f shown in FIG. 5. However, in another embodiment, the undefined pattern code can include no component. Then, in step S720, an insulating layer on the conductive layer is provided. The insulating layer exposes a part of the conductive layer to define a reaction area with an opening. Then, in step S730, a reaction layer with the ability to identify specified organism material or signal is disposed on the reaction area. Then, in step S740, a cover is disposed on the insulating layer, and the cover at least covers the reaction area. In step S750, a cutting or a punching process is performed to produce a plurality of biochemical test strips with defined pattern code. The defined pattern code partly penetrates the insulating substrate. Specifically, the defined pattern code includes a groove or a hole feature (such as the component 428b shown in FIG. 4 or the components 528b and 528e shown in FIG. 5) formed in step S750.

The pattern code of the present invention is provided for identification and to designate the data built in the measurement device. That is, one of the plurality of testing parameters, detection modes, or other information corresponding to the pattern code of the biochemical test strip can be selected for the measurement device to perform the test procedure without additional code card. The biochemical test system disclosed in the present invention not only achieves the goal to avoid the use of code card, but also reduces the production cost.

The above illustration is for a preferred embodiment of the present invention, is not limited to the claims of the present invention. Equivalent amendments and modifications without departing from the spirit of the invention should be included in the scope of the following claims.

1. A biochemical test system, comprising:
a biochemical test strip, comprising an insulating substrate, an electrode system disposed on the insulating substrate, and a pattern code disposed on one side of the insulating substrate, wherein the pattern code comprises N components, and at least one of the N components penetrates the insulating substrate, wherein N is a positive integer; and
a measurement device, comprising a microprocessor and a connector, wherein the connector is coupled to the pattern code and the electrode system for receiving a signal corresponding to the pattern code, and the microprocessor is coupled to the connector for receiving the signal from the connector.

2. The biochemical test system according to claim 1, wherein a plurality set of correction parameters are built in the microprocessor, and the microprocessor selects one set of the correction parameters to calibrate the biochemical test system according to the received signal.

3. The biochemical test system according to claim 1, wherein a plurality of modes are built in the microprocessor, and the microprocessor selects one mode for execution according to the received signal.

4. The biochemical test system according to claim 1, wherein the electrode system comprises a working electrode, a reference electrode, and a sense electrode, insulated from one another, wherein the sense electrode is provided for detecting an electrical connection between the biochemical test strip and the measurement device.

5. A measurement device, for use with a biochemical test strip, the biochemical test strip comprising an insulating substrate, an electrode system disposed on the insulating substrate, and a pattern code disposed on one side of the insulating substrate, the pattern code comprising N components, and at least one of the N components penetrating the insulating substrate, wherein N is a positive integer, the measurement device comprising:
a connector, electrically coupled to the pattern code, for receiving a signal corresponding to the component of the pattern code penetrating the insulating substrate; and
a microprocessor, coupled to the connector, for receiving the signal from the connector.

6. A biochemical test strip, comprising:
an insulating substrate;
an electrode system disposed on the insulating substrate; and
a pattern code disposed on one side of the insulating substrate;
wherein the pattern code comprises N components, N is a positive integer, and an identification code for the biochemical test strip is decided according to the location of at least one of the N components penetrating the insulating substrate.

7. The biochemical test strip according to claim 6, wherein the N components generate 2^{N}-1 identification codes.

8. The biochemical test strip according to claim 6, wherein the component penetrating the insulating substrate is a hole.

9. The biochemical test strip according to claim 6, wherein the component penetrating the insulating substrate is a groove.

10. The biochemical test strip according to claim 6, further comprising:
an insulating layer, covering a part of the electrode system, wherein a part of the electrode system uncovered by the insulating layer defines a reaction area with an opening; and
a cover, disposed on the insulating layer, for covering the reaction area, the cover having a vent corresponding to the reaction area.

11. The biochemical test strip according to claim 10, further comprising a reaction layer disposed in the reaction area, wherein the reaction layer comprises an oxidoreductase.

12. The biochemical test strip according to claim 6, wherein the electrode system comprises a working electrode, a reference electrode, and a sense electrode, insulated from one another.

13. The biochemical test strip according to claim 11, wherein the reaction layer at least covers the working electrode.

14. The biochemical test strip according to claim 6, wherein the electrode system or the pattern code comprises a material selected from a group consisting of carbon paste, silver paste, copper paste, gold-silver paste, carbon-silver paste, and a combination thereof.

15. A method for producing a biochemical test strip, comprising:
providing an insulating substrate;
forming a conductive layer on the insulating substrate;
forming an insulating layer on the conductive layer, wherein the insulating layer exposes a part of the conductive layer to define a reaction area with an opening;
providing a cover on the insulating layer, wherein the cover at least covers the reaction area; and
performing a cutting or a punching process to produce a biochemical test strip with a pattern code, wherein the pattern code comprises at least one component penetrating the insulating substrate.

16. The method according to claim 15, wherein forming the conductive layer further comprises:
providing a conductive material on the insulating substrate;
patterning the conductive material to form a working electrode, a reference electrode, a sense electrode, and a component of the pattern code, insulated from one another.

17. The method according to claim 15, wherein performing the cutting or the punching process to remove a portion of the insulating substrate to form the pattern code with the component penetrating the insulating substrate.

18. The method according to claim 15, wherein the component penetrating the insulating substrate is a hole or a groove.
